(19) United States
(12) Patent Application Publication
(10) Pub. No.: US 2025/0221918 A1
(43) Pub. Date: Jul. 10, 2025

(54) ESSENTIAL OIL COMPOSITION, MANUFACTURING METHOD THEREFOR, AND USE THEREOF IN PREPARATION OF COSMETIC PRODUCTS FOR SKIN CONDITIONS

(71) Applicant: MOTECQ CORPORATION, Taipei City (TW)

(72) Inventor: Shu-Pin HSIEH, Taoyuan City (TW)

(21) Appl. No.: 19/009,572

(22) Filed: Jan. 3, 2025

(30) Foreign Application Priority Data
Jan. 5, 2024 (TW) 113100659

(51) Int. Cl. A61K 8/92; A61K 8/35; A61K 8/64; A61K 8/73; A61K 8/891; A61K 8/98; A61Q 19/00

(52) U.S. Cl. CPC A61K 8/922; A61K 8/355; A61K 8/64; A61K 8/735; A61K 8/891; A61K 8/987; A61Q 19/00

(57) ABSTRACT

An essential oil composition, at least including a silicone mixture and a vegetable oil mixture, wherein the silicone mixture includes a first polysiloxane and a second polysiloxane, the vegetable oil mixture includes an emollient formed by at least three vegetable oils, and the vegetable oil mixture is added dropwise to the silicone mixture while the silicone mixture is being stirred. The essential oil composition is transparent and non-turbid in appearance, and has a refreshing effect after being applied to skin surface. After washing hands, water droplets would not be present on the skin surface which has the composition applied thereto, and therefore would not feel sticky or greasy. The present invention further provides a method for manufacturing an essential oil composition, and a use of an essential oil composition in preparation of a cosmetic product for improving skin conditions, thereby relieve the red and swollen appearance of dermatitis.

### BACKGROUND OF THE INVENTION

### a) Field of the Invention

The present invention relates to an essential oil composition, and more particularly to an essential oil composition which is transparent and non-turbid in appearance and applicable to skin, and a manufacturing method therefor, such that a refreshing effect is produced on skin surface after the essential oil composition is applied to the skin surface, and no water droplets are present on the skin surface which has the composition applied thereto, and therefore would not feel greasy. The present invention further relates to a use of an essential oil composition in preparation of a cosmetic product for improving skin conditions, and more particularly to local applications of a cosmetic product, such that the red and swollen appearance of atopic dermatitis can be relieved.

### b) Description of the Prior Art

The main ingredient of a traditional essential oil is an emollient, and the effect of the emollient is to form an oil layer on skin surface, so as to lock moisture in the skin, soften and moisturize the skin at the same time, protect the skin from irritations, and therefore reduce skin itches and stratum corneum exfoliation, for instance, the traditional essential oil composition in PCT publication number WO2014167552A1 (referred to as Reference Document 1 hereafter).

However, Reference Document 1 requires the use of an emulsifier/thickener, which is because the emollient included in Reference Document 1 uses a vegetable oil, a mineral oil and/or an organic silicone oil (which may also be called polysiloxane, silicone or siloxane), and a large amount of water. Therefore, the composition of Reference Document 1 is an emulsion and is turbid and non-transparent in appearance. The use of an emulsifier/thickener in a composition increases the risk of individual skin disorders, whereas a non-transparent composition raises doubts of consumers and negatively influences sales.

In Chinese Patent No. CN108014028A (referred to as Reference Document 2 hereafter), water, emulsifiers and thickeners are not used, but instead uses a mixture of a high-viscosity polydimethylsiloxane and a low-viscosity silicone oil, and then combines same with Hippophae rhamnoides fruit oil/bisabolol (a vegetable oil) and other ingredients by mixing and stirring, so as to obtain a non-sticky, uniformly transparent and non-turbid composition. Although Reference Document 2 claims a transparent composition can be obtained, Reference Document 2 only gives the example of using two vegetable oils. Therefore, regarding an essential oil composition which uses more than two vegetable oils, it would be doubtful as to whether an essential oil composition will remain “non-sticky and uniformly transparent and non-turbid in appearance” after the same is subjected to combining by mixing and stirring. In addition, in the case that the ingredients constituting an essential oil composition inevitably include water or only a very small amount of water on the basis of a particular requirement, since Reference Document 2 only discloses combining by mixing and stirring suitable under anhydrous conditions, Reference Document 2 apparently does not provide an effective mode of combining, such that the effect of “non-sticky, uniformly transparent and non-turbid in appearance” can be maintained.

Moreover, skin surface is easily susceptible to problems such as itchiness, swelling and pains due to allergies and aging resulted from external environmental influences on skin, and since a cosmetic product is not a medication, the above-mentioned problems usually cannot be improved by applying the cosmetic product. However, once a medication is used, the applied area and other nearby areas on skin cannot have cosmetic products further applied thereto, which is a very inconvenient and difficult dilemma to people who have to use cosmetic products.

### SUMMARY OF THE INVENTION

Therefore, the object of the present invention is to provide an essential oil composition which does not use an emulsifier and a thickener, the essential oil composition does not include water or only a small amount of water, and the essential oil composition includes more than two vegetable oils. The present invention further provides a manufacturing method of an essential oil composition, the essential oil composition manufactured by using the manufacturing method of the essential oil composition is transparent and non-turbid in appearance, has a refreshing effect after being applied to skin surface, and after washing hands, water droplets would not be present on the skin surface which has the composition applied thereto, and therefore would not feel sticky or greasy. The present invention further provides a use of an essential oil composition in preparation of a cosmetic product for improving skin conditions, so as to relieve the red and swollen appearance of atopic dermatitis.

Particularly, the symbol “-” used in the description and the claims indicates that the value range refers to a range which has the values recited before and after the “-” symbol as the lower limit and the upper limit, and the “wt %” used in the description and the claims refers to weight percentage.

An essential oil composition provided according to the above-mentioned objects, at least comprising: a silicone mixture and a vegetable oil mixture, based on the essential oil composition being 100 wt %, the silicone mixture being 70 wt %-94.01 wt %, wherein the silicone mixture comprises cyclopentasiloxane and polydimethylsiloxane (or dimethicone), the vegetable oil mixture comprises an emollient formed by at least three vegetable oils, more preferably, the emollient is formed by at least three selected from a group consisting of Sclerocarya birrea seed oil, Prunus amygdalus dulcis oil, Helianthus annuus seed oil and Argania spinosa kernel oil.

According to the above-mentioned essential oil composition, the emollient comprises Sclerocarya birrea seed oil, Prunus amygdalus dulcis oil, Helianthus annuus seed oil and Argania spinosa kernel oil.

According to the above-mentioned essential oil composition, the essential oil composition does not comprise water, emulsifiers and/or thickeners, or the essential oil composition does not comprise emulsifiers and/or thickeners, but comprise no more than 0.010195 wt % of water.

According to the above-mentioned essential oil composition, the polydimethylsiloxane in the silicone mixture has a viscosity of below 7000 cps at 25° C.

A manufacturing method of an essential oil composition provided according to the above-mentioned objects of the invention, which is used to manufacture the above-mentioned essential oil composition, the manufacturing method of the essential oil composition comprises the following steps: a) a silicone mixture manufacturing step, adding a first polysiloxane to a second polysiloxane by means of a silicone mixture titration mode, so as to form the silicone mixture; b) a vegetable oil mixture manufacturing step, combining an emollient formed by at least three vegetable oils to form the vegetable oil mixture, more preferably, the vegetable oil mixture is formed by combining the emollient formed by at least three selected from a group consisting of Sclerocarya birrea seed oil, Prunus amygdalus dulcis oil, Helianthus annuus seed oil and Argania spinosa kernel oil.; and c) an essential oil composition manufacturing step, adding the vegetable oil mixture to the silicone mixture by means of an essential oil composition titration mode, so as to form the essential oil composition.

According to the above-mentioned manufacturing method of an essential oil composition, the silicone mixture titration mode is adding the first polysiloxane dropwise to the second polysiloxane while the second polysiloxane is being stirred.

According to the above-mentioned manufacturing method of an essential oil composition, the first polysiloxane is the cyclopentasiloxane and the second polysiloxane is the polydimethylsiloxane, or the second polysiloxane is the cyclopentasiloxane and the first polysiloxane is the polydimethylsiloxane.

According to the above-mentioned manufacturing method of an essential oil composition, the essential oil composition titration mode is adding the vegetable oil mixture dropwise to the silicone mixture while the silicone mixture is being stirred.

According to the above-mentioned essential oil composition and the manufacturing method of an essential oil composition, the vegetable oil mixture further comprises a skin conditioning agent and an accompanying supplementary agent thereof, the skin conditioning agent being formed by at least one selected from a group consisting of hyaluronic acid, tocopheryl acetate (Vitamin E), ubiquinone (Q10), sturgeon caviar extract, fibroin extracted from silkworm cocoons and bee venom produced by bees; in addition, in the vegetable oil mixture manufacturing step, the emollient is combined with the skin conditioning agent and an accompanying supplementary agent thereof to form the vegetable oil mixture. The supplementary agent is used to assistively enable the skin conditioning agent to be formulated into an adequate concentration for ease of use, or the supplementary agent can add scents, the supplementary agent being, for instance, a fragrance, water, butanediol, phenoxyethanol, honey, ethylhexylglycerin, caprylyl glycol, hexanediol or ethylhexyl palmitate.

According to the above-mentioned essential oil composition and the manufacturing method of an essential oil composition, based on the essential oil composition being 100 wt %, the essential oil composition at least comprises: 70 wt %-93.9 w t% of the silicone mixture, wherein the cyclopentasiloxane is 60 wt %-89.51 wt %, the polydimethylsiloxane is 4.39 wt %-12.74 wt %; Sclerocarya birrea seed oil of the emollient being 2 wt %-5 wt %; a sum of Prunus amygdalus dulcis oil and Helianthus annuus seed oil of the emollient being 1.5 wt %-4 wt %; Argania spinosa kernel oil of the emollient being 1 wt %-4 wt %; hyaluronic acid of the skin conditioning agent being 0.48 wt %-0.6 wt %; tocopheryl acetate of the skin conditioning agent being 1 wt %-3 wt %; ubiquinone of the skin conditioning agent being 0.01 wt %-0.05 wt %; sturgeon caviar extract of the skin conditioning agent being 0.002 wt %-0.005 wt %; fibroin extracted from silkworm cocoons of the skin conditioning agent being 0.006 wt %-0.01wt %; bee venom produced by bees of the skin conditioning agent being 0.00005 wt %-0.005 wt %; and the remainder being made up by an supplementary agent, the supplementary agent being anhydrous or the supplementary agent comprising no more than 0.010195 wt % of water.

According to the above-mentioned essential oil composition and the manufacturing method of an essential oil composition, based on the essential oil composition being 100 wt %, the essential oil composition at least comprises: 93.9 wt % of the silicone mixture, the emollient of the vegetable oil mixture being 4.5 wt %, the skin conditioning agent being 1.49805 wt %, and the remainder being made up by the supplementary agent accompanying the skin conditioning agent, wherein in the silicone mixture, the cyclopentasiloxane is 89.51 wt % and the polydimethylsiloxane is 4.39 wt %; in the emollient, Sclerocarya birrea seed oil is 2 wt %, a sum of Prunus amygdalus dulcis and Helianthus annuus seed oil is 1.5 wt %, and Argania spinosa kernel oil is 1 wt %; in the skin conditioning agent, hyaluronic acid is 0.48 wt %, tocopheryl acetate is 1 wt %-3 wt %, ubiquinone is 0.01 wt %, sturgeon caviar extract is 0.002 wt %, fibroin extracted from silkworm cocoons is 0.006 wt %, and bee venom produced by bees is 0.00005 wt %, the supplementary agent is anhydrous or the supplementary agent comprises no more than 0.010195 wt % of water.

According to the above-mentioned essential oil composition and the manufacturing method of an essential oil composition, based on the essential oil composition being 100 wt %, the essential oil composition at least comprises: 84.93 wt % of the silicone mixture, 12 wt % of the emollient of the vegetable oil mixture, 1.503 wt % of the skin conditioning agent, and the remainder being made up by the supplementary agent accompanying the skin conditioning agent, wherein in the silicone mixture, the cyclopentasiloxane is 72.19 wt % and the polydimethylsiloxane is 12.74 wt %; in the emollient, Sclerocarya birrea seed oil is 4 wt %, a sum of Prunus amygdalus dulcis and Helianthus annuus seed oil is 4 wt %, and Argania spinosa kernel oil is 4 wt %; in the skin conditioning agent, hyaluronic acid is 0.48 wt %, tocopheryl acetate is 1 wt %-3 wt %, ubiquinone is 0.01 wt %, sturgeon caviar extract is 0.002 wt %, fibroin extracted from silkworm cocoons is 0.006 wt %, and bee venom produced by bees is 0.005 wt %; the supplementary agent is anhydrous or the supplementary agent comprises no more than 0.010195 wt % of water.

According to the above-mentioned essential oil composition and the manufacturing method of an essential oil composition, the essential oil composition comprising: the silicone mixture and the vegetable oil mixture, wherein the silicone mixture comprises a first polysiloxane and a second polysiloxane, and the first polysiloxane and the second polysiloxane are different polysiloxanes; the vegetable oil mixture comprises an emollient formed by at least three vegetable oils, and the vegetable oil mixture is added dropwise to the silicone mixture while the silicone mixture is being stirred.

The present invention further provides a use of the above-mentioned essential oil composition in preparation of a cosmetic product for improving skin conditions.

### BRIEF DESCRIPTION OF THE DRAWINGS

FIG. 1 is a picture showing the transparent appearance of an essential oil composition of Embodiment 2 in a beaker.

FIG. 2 is a picture showing the turbid appearance of an essential oil composition of Comparative Example 1 in a beaker.

FIG. 3 are pictures showing the red and swollen appearance of facial skin suffering from atopic dermatitis, and the improved skin condition after applying Embodiment 3.

### DETAILED DESCRIPTION OF THE PREFERRED EMBODIMENTS

The following embodiments and comparative examples are provided to facilitate better understanding of the technical contents, characteristics and effects of the present invention by the examiner. It is to be understood that the present invention is not limited by the embodiments disclosed herein.

The following ingredients of corresponding wt % are prepared according to Table 1 below.

TABLE 1
The Ingredients of an essential oil composition and proportions thereof.

| Ingredients of an | Embodiment | Embodiment | Embodiment | Embodiment | Comparative |
|---|---|---|---|---|---|
| essential oil | 1 | 2 | 3 | 4 | example 1 |
| composition | (wt %) | (wt %) | (wt %) | (wt %) | (wt %) |
| Cyclopentasiloxane | 60 | 89.51 | 72.19 | 89.6 | 89.51 |
| Polydimethylsiloxane | 10 | 4.39 | 12.74 | 4.41 | 4.39 |
| Sclerocarya birrea oil | 5 | 2 | 4 | 2 | 2 |
| Prunus amygdalus | 4 | 1.5 | 4 | 1.5 | 1.5 |
| dulcis oil and | | | | | |
| Helianthus annuus | | | | | |
| seed oil | | | | | |
| Argania spinosa | 3 | 1 | 4 | 1 | 1 |
| kernel oil | | | | | |
| Hyaluronic acid | 0.6 | 0.48 | 0.48 | 0.48 | 0.48 |
| Tocopheryl acetate | 3 | 1 | 1 | 1 | 1 |
| Ubiquinone | 0.05 | 0.01 | 0.01 | 0.01 | 0.01 |
| Sturgeon caviar | 0.005 | 0.002 | 0.002 | 0 | 0.002 |
| extract | | | | | |
| Fibroin extracted | 0.01 | 0.006 | 0.006 | 0 | 0.006 |
| from silkworm | | | | | |
| cocoons | | | | | |
| Bee venom | 0.001 | 0.00005 | 0.005 | 0 | 0.00005 |
| produced by bees | | | | | |
| Supplementary | Remainder | Remainder | Remainder | 0 | Remainder |
| agent | | | | | |
| Preparation Mode | Titration | Titration | Titration | Titration | Mixing and |
| | | | | | stirring |

### Manufacturing the Silicone Mixtures of Embodiments 1-3 According to the Silicone Mixture Manufacturing Step

Adding a first polysiloxane to a second polysiloxane by means of a silicone mixture titration mode, so as to form a silicone mixture, the silicone mixture titration mode being adding the first polysiloxane dropwise to the second polysiloxane while the second polysiloxane is being stirred, the second polysiloxane being cyclopentasiloxane and the first polysiloxane being polydimethylsiloxane. In other words, the weighed and ready cyclopentasiloxane is placed into a beaker and continuously stirred by using a magnetic stirrer, the weighed and ready polydimethylsiloxane is loaded into a burette and provided above the above-mentioned beaker, and then the polydimethylsiloxane is added dropwise to the cyclopentasiloxane by titration, until all of the polydimethylsiloxane are added to the beaker having the cyclopentasiloxane.

### Manufacturing the Vegetable Oil Mixtures of Embodiments 1-3 According to the Vegetable Oil Mixture Manufacturing Step

Simultaneously or in batches, loading the weighed and ready and converted Sclerocarya birrea seed oil, Prunus amygdalus dulcis oil, Helianthus annuus seed oil and Argania spinosa kernel oil of the emollient, and hyaluronic acid, tocopheryl acetate, ubiquinone, sturgeon caviar extract, fibroin extracted from silkworm cocoons and bee venom produced by bees of the skin conditioning agent into a beaker, and stirring same by using a magnetic stirrer to enable uniform mixing, wherein the supplementary agent is from ingredients included in commercially sold hyaluronic acid, sturgeon caviar extract, fibroin extracted from silkworm cocoons and bee venom produced by bees.

### Manufacturing the Essential Oil Composition of Embodiments 1-3 According to the Essential Oil Composition Manufacturing step

Adding the vegetable oil mixture to the silicone mixture by means of an essential oil composition titration mode, so as to form the essential oil composition, and the essential oil composition titration mode being adding the vegetable oil mixture dropwise to the silicone mixture while the silicone mixture is being stirred. In other words, the weighed and ready silicone mixture is placed into a beaker and continuously stirred by using a magnetic stirrer, the weighed and ready vegetable oil mixture is loaded into a burette and provided above the above-mentioned beaker, and then the vegetable oil mixture is added dropwise to the silicone mixture by titration, until all of the vegetable oil mixture is added to the beaker having the silicone mixture.

### Manufacturing the Essential Oil Composition of Embodiment 4

The essential oil composition of Embodiment 4 is performed in a way similar to the above-mentioned modes for preparing Embodiments 1-3, apart from not using sturgeon caviar extract, fibroin extracted from silkworm cocoons, bee venom produced by bees and ingredients of a supplementary agent, the remaining parts are the same as the modes for preparing Embodiments 1-3, and will not be repeated here.

### Manufacturing the Essential Oil Composition of Comparative Example 1

The ingredients of Comparative Example 1 are the same as those of Embodiment 2, but Comparative Example 1 uses the mixing and stirring mode of Reference Document 2. In other words, all the ingredients are mixed together and then stirred (for instance, by magnetic stirring or stirring by blades of a stirrer), instead of the titration mode used in Embodiment 2.

### Effect Testing

Transparency testing: loading an essential oil composition in a transparent glass bottle and leaving to stand for 24 hours, observing the transparency thereof by naked eyes, a transparent result means good transparency, while a turbid result means poor transparency.

Refreshingness testing: spreading an essential oil composition on skin surface and observing after 30 seconds, if no residues of the essential oil composition is observed, marking same as being refreshing; if a residue of the essential oil composition is observed, marking same as not refreshing.

Non-greasy testing: spreading an essential oil composition on skin surface and observing after 30 seconds, if the skin does not feel sticky by touching the applied area, and no water droplets are present on the applied skin surface after washing hands, marking same as being non-greasy, if the skin feels sticky by touching the applied area, or water droplets are present on the applied skin surface after washing hands, marking same as being greasy.

The results of the above-mentioned transparency testing, refreshingness testing and non-greasy testing are recorded in Table 2 below.

TABLE 2

| | | | | | Comparative |
|---|---|---|---|---|---|
| Effect Testing | Embodiment 1 | Embodiment 2 | Embodiment 3 | Embodiment 4 | example 1 |
| Transparency | Transparent | Transparent | Transparent | Transparent | Turbid |
| testing | | | | | |
| Refreshingness | Refreshing | Refreshing | Refreshing | Refreshing | Not |
| testing | | | | | refreshing |
| Non-greasy | Non-greasy | Non-greasy | Non-greasy | Non-greasy | Greasy |
| testing | | | | | |
| Overall | Qualified | Qualified | Qualified | Qualified | Not qualified |
| evaluation | | | | | |

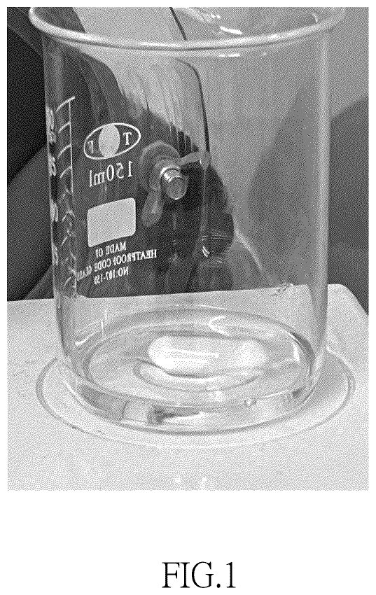

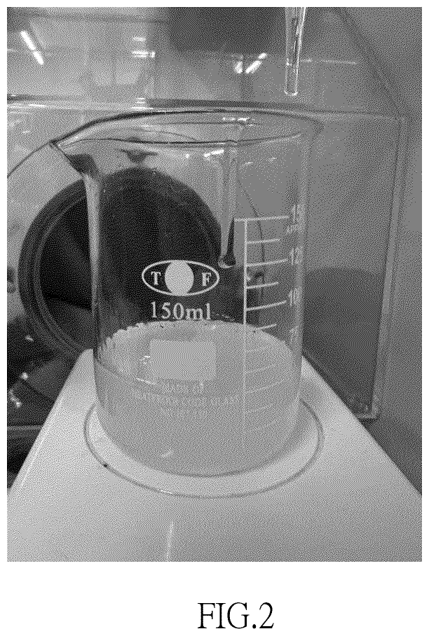

It can be seen from Table 1 and Table 2 that, in particular, the difference between Embodiment 2 and Comparative Example 1 lies in that the manufacturing method of the essential oil composition used in Embodiment 2 is titration mode and the essential oil composition is transparent in appearance. Refer to FIG. 1, the transparent liquid in the beaker is the essential oil composition of Embodiment 2, whereas Comparative Example 1 uses the mixing and stirring mode of Reference Document 2 and the composition is turbid in appearance, as can be seen in FIG. 2, the turbid liquid in the beaker is the essential oil composition of Comparative Example 1. Therefore, this indicates the titration mode used in the present invention is the main reason that the formed essential oil composition being transparent in appearance.

In addition, both Comparative Example 1 and Reference Document 2 use the mixing and stirring mode, and the major difference between them being that Comparative Example 1 uses four vegetable oils, whereas Reference Document 2 only uses two vegetable oils. Obviously, the mixing and stirring mode is inadequate for using more than two vegetable oils to manufacture an essential oil composition.

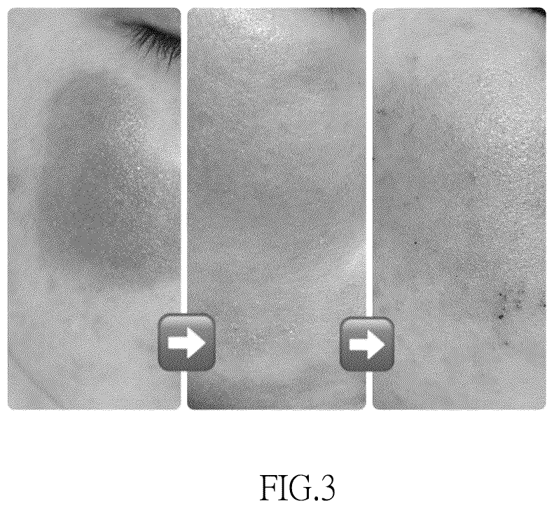

Moreover, the present invention further provides a use of an essential oil composition in preparation of a cosmetic product for improving skin conditions, which uses the essential oil compositions of Embodiments 1-3 as cosmetic products for spreading on local surfaces of the skin, so as to improve skin conditions. For instance, for improving skin allergies and aging caused by the environment, as well as problems such as itchiness, swelling and pains of skin surface resulted from external environmental influences, circadian rhythm disorders or congenital sensitive skin. Further, the essential oil compositions can relieve atopic dermatitis, red and swollen allergies, and speed up microtrauma repairing. In FIG. 3, the picture on the left shows the red and swollen appearance of facial skin suffering from atopic dermatitis. 1 g of the essential oil composition of Embodiment 3 was obtained and then used as a cosmetic product for being spread on a local surface of the skin which showed the red and swollen appearance, which covered the skin as a thin film. After 1 day, the red and swollen appearance had diminished, which indicates that the skin condition was improved, as shown by the picture in the middle of FIG. 3. Subsequently, 1 g of the essential oil composition of Embodiment 3 was continuously spread on the skin area showing the diminished red and swollen appearance, after another day, the red and swollen appearance became even more diminished, which indicates that the skin condition was even more improved, as shown in the picture on the right of FIG. 3. Therefore, Embodiment 3 is the most preferred embodiment.

In summary, the manufacturing method of the essential oil composition of the present invention uses the titration mode, therefore, when more than two vegetable oils are used, the essential oil composition has the characteristics of being transparent, and is refreshing and non-sticky after being applied to skin. In addition, the present invention further provides a use of an essential oil composition in preparation of a cosmetic product for improving skin conditions, which uses the essential oil composition as a cosmetic product for spreading on local surfaces of the skin, so as to improve skin conditions.

It should be understood that the above-mentioned features are merely embodiments of the present invention and are not to be used to limit the scope of the present invention. Any simple changes and modifications which are effectually equivalent and made according to the claims and the description of the present invention still fall within the scope of the claims of the present invention.

What is claimed is:

1. An essential oil composition, at least comprising: a silicone mixture and a vegetable oil mixture, based on the essential oil composition being 100 wt %, the silicone mixture being 70 wt %-94.01 wt %, wherein the silicone mixture comprises a first polysiloxane and a second polysiloxane, and the first polysiloxane and the second polysiloxane are different polysiloxanes, the vegetable oil mixture comprises an emollient formed by at least three vegetable oils, and the vegetable oil mixture is added dropwise to the silicone mixture while the silicone mixture is being stirred.

2. The essential oil composition according to claim 1, wherein the essential oil composition does not comprise water, or the essential oil composition comprises no more than 0.010195 wt % of water.

3. The essential oil composition according to claim 2, wherein the vegetable oil mixture further comprises a skin conditioning agent, the skin conditioning agent is formed by at least one selected from a group consisting of hyaluronic acid, tocopheryl acetate, ubiquinone, sturgeon caviar extract, fibroin extracted from silkworm cocoons and bee venom produced by bees.

4. The essential oil composition according to claim 3, wherein the first polysiloxane is cyclopentasiloxane and the second polysiloxane is polydimethylsiloxane.

5. The essential oil composition according to claim 4, wherein the emollient of the vegetable oil mixture is formed by at least three selected from a group consisting of Sclerocarya birrea seed oil, Prunus amygdalus dulcis oil, Helianthus annuus seed oil and Argania spinosa kernel oil.

6. The essential oil composition according to claim 5, wherein based on the essential oil composition being 100 wt %, the essential oil composition at least comprises: 70 wt %-93.9 wt % of the silicone mixture, wherein the cyclopentasiloxane is 60 wt %-89.51 wt %, the polydimethylsiloxane is 4.39 wt %-12.74 wt %; the Sclerocarya birrea seed oil of the emollient is 2 wt %-5 wt %, a sum of the Prunus amygdalus dulcis oil and the Helianthus annuus seed oil of the emollient is 1.5 wt %-4 wt %, the Argania spinosa kernel oil of the emollient is 1 wt %-4 wt %; the hyaluronic acid of the skin conditioning agent is 0.48 wt %-0.6 wt %, the tocopheryl acetate of the skin conditioning agent is 1 wt %-3 wt %, the ubiquinone of the skin conditioning agent is 0.01 wt %-0.05 wt %, the sturgeon caviar extract of the skin conditioning agent is 0.002 wt %-0.005 wt %, the fibroin extracted from silkworm cocoons of the skin conditioning agent is 0.006 wt %-0.01 wt %, the bee venom produced by bees of the skin conditioning agent is 0.00005 wt %-0.005 wt %; and a remainder is made up by an supplementary agent, the supplementary agent being anhydrous or the supplementary agent comprising no more than 0.010195 wt % of water.

7. A use of the essential oil composition according to claim 1 in preparation of a cosmetic product for improving skin conditions.

8. A manufacturing method of an essential oil composition, used for manufacturing an essential oil composition, the manufacturing method of the essential oil composition comprises the following steps of: a) a silicone mixture manufacturing, adding a first polysiloxane to a second polysiloxane by means of a silicone mixture titration mode, hereby forming a silicone mixture; b) a vegetable oil mixture manufacturing, combining an emollient formed by at least three vegetable oils to form the vegetable oil mixture; and c) an essential oil composition manufacturing, adding the vegetable oil mixture to the silicone mixture by means of an essential oil composition titration mode, hereby forming the essential oil composition.

9. The manufacturing method of an essential oil composition according to claim 8, wherein the silicone mixture titration mode is adding the first polysiloxane dropwise to the second polysiloxane while the second polysiloxane is being stirred; the essential oil composition titration mode is adding the vegetable oil mixture dropwise to the silicone mixture while the silicone mixture is being stirred.

10. The manufacturing method of an essential oil composition according to claim 9, wherein the first polysiloxane is cyclopentasiloxane and the second polysiloxane is polydimethylsiloxane, or the second polysiloxane is cyclopentasiloxane and the first polysiloxane is polydimethylsiloxane.

11. The manufacturing method of an essential oil composition according to claim 10, wherein the emollient of the vegetable oil mixture is formed by at least three selected from a group consisting of Sclerocarya birrea seed oil, Prunus amygdalus dulcis oil, Helianthus annuus seed oil and Argania spinosa kernel oil.

12. The manufacturing method of an essential oil composition according to claim 11, wherein the vegetable oil mixture further comprises a skin conditioning agent, and the skin conditioning agent is formed by at least one selected from a group consisting of hyaluronic acid, tocopheryl acetate, ubiquinone, sturgeon caviar extract, fibroin extracted from silkworm cocoons and bee venom produced by bees.
